(19) United States
(12) Patent Application Publication
(10) Pub. No.: US 2024/0324914 A1
(43) Pub. Date: Oct. 3, 2024

(54) Pulse Oximeter

(71) Applicant: Nicholas Lazares, Milton, MA (US)

(72) Inventor: Nicholas Lazares, Milton, MA (US)

(21) Appl. No.: 18/191,073

(22) Filed: Mar. 28, 2023

(51) Int. Cl. A61B 5/1455; A61B 5/00

(52) U.S. Cl. CPC A61B 5/14552; A61B 5/6898; A61B 2560/0214

(57) ABSTRACT

A smart phone case is provided having a built in pulse oximeter. The pulse oximeter may operate independently of the smartphone or along with the smartphone. The present disclosure allows users to have access to a pulse oximeter to measure blood oxygen concentration on the go in a convenient location without needing to keep track of a separate device. Studies continue to show the importance of blood oxygen concentration monitoring for tracking respiratory disease symptoms, lung function at altitude, and heart function, among others.

### BACKGROUND OF THE INVENTION

The present disclosure relates generally to pulse oximeters. More particularly the present disclosure relates to a device having a pulse oximeter connected to or integrated into a protective case of a cell phone or other mobile device.

Monitoring blood oxygen levels is a low cost and non-invasive way to track how an individual is breathing and/or tolerating high elevations. It can be an indicator of many problems such as ineffective lung function, ineffective heart function, when a disease such as a respiratory infection becomes concerning, and how a person is tolerating high altitude. Further, low oxygen levels are considered a key predictor of poor outcomes for COVID-19 patients. Lowered oxygen levels can even occur before the onset of symptoms of COVID-19 and other respiratory diseases.

Thus, it can be very important, and convenient, for everyday users to monitor their own blood oxygen levels. In the prior art, a stand alone, battery powered device is often used which relies on optical sensing of blood in the finger. While these work in some instances, the small nature of the devices leads to them being easily misplaced and unavailable when needed. Also, they are inconvenient for consistent oxygen saturation monitoring because it becomes just another item that has to be carried around with a person along with other sundry items, further leading it to be forgotten, lost, and the like.

Therefore, what is needed is a device for monitoring blood oxygen levels that is conveniently located on a device that is often carried with someone—such as a cellular telephone case.

### SUMMARY OF THE INVENTION

The subject matter of this application may involve, in some cases, interrelated products, alternative solutions to a particular problem, and/or a plurality of different uses of a single system or article.

In one aspect, a case for a smartphone is provided. The case has a rear wall for covering at least a portion of the smartphone when placed therein, and a sidewall portion which extends form the rear wall to cover at least a part of an edge of the smartphone and to removably engage the case with the smartphone. A pulse oximeter is connected do and/or integrated with the rear wall of the case an in communication with a display on the case, or in communication with a smartphone via wired or wireless communication to use the smartphone to display readouts gathered from the pulse oximeter. Some users may prefer to have their health data stored on their own device and not online for privacy reasons. In some embodiments, the measured health data may not be stored at all.

In another aspect, a method for measuring blood oxygen concentration is provided. The method involves activating a pulse oximeter which is stored on or in a smartphone case. The method further involves receiving a finger placed in or on the pulse oximeter and holding it still while the device takes a reading. Finally, the method involves presenting, on a display, information corresponding to a blood oxygen concentration recorded by the pulse oximeter.

### BRIEF DESCRIPTION OF DRAWINGS

FIG. 1 provides an elevation view of an embodiment of the present disclosure.

FIG. 2 provides a side rear view of an embodiment of the present disclosure.

FIG. 3 provides a perspective view of another embodiment of the present disclosure.

FIG. 4 provides a perspective view of yet another embodiment of the present disclosure.

FIG. 5 provides a view of a computerized user interface to track data gathered by the oxygen sensor.

### DETAILED DESCRIPTION OF THE INVENTION

The detailed description set forth below in connection with the appended drawings is intended as a description of presently preferred embodiments of the invention and does not represent the only forms in which the present disclosure may be constructed and/or utilized. The description sets forth the functions and the sequence of steps for constructing and operating the invention in connection with the illustrated embodiments.

Generally, the present disclosure concerns a cellular telephone case which comprises a sensor which can determine, among other things, pulse and blood oxygen saturation. The sensor and related components are referred to herein generally as a pulse oximeter. In varying embodiments, the pulse oximeter may stand alone, having its own battery, processor, memory for data storage, display, and sensor(s). In other embodiments, the pulse oximeter may be in communication with a smartphone held in the case, allowing it to utilize one or more of a battery, charger or charge port, processor, memory, and/or display. In a particular embodiment, the pulse oximeter may be completely self-contained without a data transfer to any device to ensure privacy and data isolation. In such an embodiment, the device may also not have a memory to store past data, such that there is no storage of any past readings.

In most embodiments, the pulse oximeter contemplated herein utilizes one or more light sources (which may include light sources emitting within the visible light spectrum and/or emitting at wavelengths on the electromagnetic spectrum outside of visible light range). Light sources may be a laser or lasers, non-coherent light, or combinations thereof. The pulse oximeter also may utilize a photodetector to receive and detect light from the light source after it has passed through or otherwise engaged with blood in tissue being tested (typically a finger tip). The photodetector may be on a surface opposite to that of the light source to detect light which has passed through the tissue positioned between light source and photodetector, or may be on the same surface as the light source to detect light as it interacts with the tissue being tested. Lensing, relative angles, and the like may also be used to improve detection and precision. The signal gathered by the photodetector is sent to a processor either via a wired or wireless connection. The processor is programmed to calculate an oxygen saturation, pulse, and optionally other outputs based on the signal received and to send it to a display to present the sensed data. As noted above, the processor may be integrated into the case, and/or may be a processor on a cellular telephone which is held by the case of the present disclosure. A memory may also be employed in communication with the processor to, among other things, store data such as history of readings, programming, and the like. The memory may be integrated into the case, on a surface of the case, and/or may be a memory of a cellular telephone which is held by the case. Operation and activation of the pulse oximeter may be done by opening a cover to automatically power on the device, by a button, touch or swipe input on a surface of the case, activation by a user interface on the smartphone, among other options.

Light sources may vary and are selected to provide a reliable, repeatable reading to the photodetector that correlates to blood oxygen concentration, typically by recording a color of the blood which correlates to the oxygen saturation (sp02%) of the blood. In many embodiments, one or a combination of a red light and infrared light source are used. The photodetector, typically placed on an opposite side of the finger on a cover, but optionally also on the same side of the finger as the light source, then reads the light passing through and/or reflecting/refracting from the blood in the tissue placed on the sensor. Using a processor programmed with an algorithm to correlate the sensed light to a blood oxygen concentration and pulse rate (among others). In other embodiments, a green light may be used in addition to the red and infrared light sources, or in alternative to one or both. The green light may be particularly useful in embodiments having the sensor on a same side of the sensor as the light source. As noted, these light sources may be laser sources, or non-coherent light sources. In a particular embodiment, the pulse oximeter may further be configured and operational to measure a blood glucose level optically as well.

In some embodiments, a cover, such as a spring loaded (or other flexible material) cover, may be used to protect the pulse oximeter as well as to house the photodetector. In other embodiments, a cover without a photodetector may be used simply to protect the components. In still other embodiments, a durable but transparent or translucent surface such as glass or a hard plastic may protect the light source and/or photodetector without the need for an opaque or removable cover.

As noted above, in one embodiment, the pulse oximeter components may be in electronic communication with a smartphone held within the cover. A processor of the smartphone may be operable to control the operation of the pulse oximeter via, for example, a user input into the user interface of the smart phone and/or automatically depending on sensed conditions by the smartphone. Further, the photosensor or other detector may be in communication with the processor of the smartphone and may be programmed with an algorithm to calculate and display parameters such as oxygen saturation and pulse rate. The photosensor is a sensor operable to measure light emitted from the light array.

In addition to the pulse oximeter components noted below, the case is formed of a rear wall and a sidewall which extends from the rear wall. The sidewall is configured to cover at least a part of an edge of the smartphone to be positioned in the case. Similarly, the rear wall is sized and shaped to cover at least a part if not all of a rear of the smartphone. The rear wall and sidewall or sidewalls together define a chamber region which is sized and configured to receive a smartphone.

In one embodiment, the smartphone case will connect electronically with the case via a communications port. In another embodiment, the case will connect with the smartphone through a communications port and may comprise a second port to serve as a relay allowing connection of a charging or data connection through the relay to the smartphone. In a particular embodiment, the case connection to the smartphone through the communications port allows a power draw from a battery of the phone to power operation of the pulse oximeter components. In another embodiment, a female communications port of the phone will be connected with a male connection of the case, with the case having a female port on an exterior to allow connectivity and charging to the smartphone case.

In one embodiment, the case may have a rechargeable battery built into its body or connected to its body. The battery, as discussed above, is operable to power the components of the pulse oximeter. In a particular embodiment, the battery is in communication with the light array to provide electricity for it to operate. The rechargeable battery may be charged in a number of manners. In one embodiment, a separate charge port may connect to a charger to charge the battery. In another embodiment, the case may split power to direct a current flow to a battery of the pulse oximeter and to a battery of a smartphone when the smartphone is in the case, from a connection of a charger with the phone via a wiring body. Thus, this wiring body allows charging of both the battery of the pulse oximeter and a battery of the smartphone at the same time. In still another embodiment, the rear wall may have a wireless charging positioned in or on it. The wireless charging coil is connected to a battery of the pulse oximeter, also in or on the case body, and is operable to charge the battery via wireless charging. In a particular embodiment, the wireless charging coil is smaller than a wireless charging coil of a smartphone intended to be placed in the case, so as to allow simultaneous wireless charging of a battery in the smartphone and the battery of the pulse oximeter.

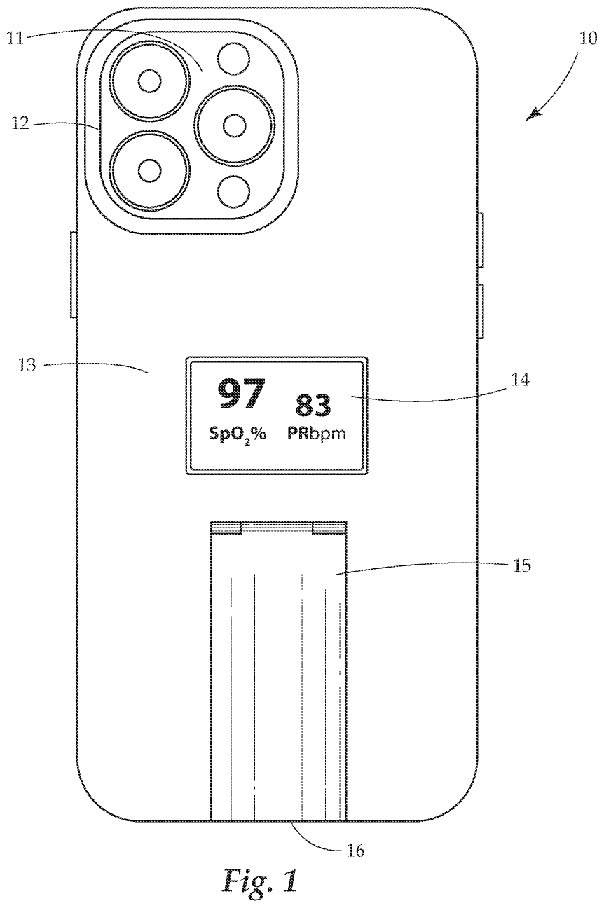

Turning now to FIGS. 1-3 elevation, side, and perspective views of an embodiment of the cellular phone case having a pulse oximeter built into it is shown. The case 10 has a rear wall 13 which defines the case body and covers at least a portion of the smartphone 11 held therein. In the embodiment shown, an opening 12 is defined around a camera assembly on the phone 11. A pulse oximeter 16 is positioned on the rear wall 13 and is accessible by a finger of a user to be placed into the pulse oximeter 16 such that a fingertip can be placed against a surface of the pulse oximeter 16, and blood oxygen, pulse, and other readings can be taken. The pulse oximeter 16 is attached to the outside of the rear wall 13. This connection to the outside of the rear wall 13 may also refer to an integrated/inset embodiment. A cover 15 is positioned on a top of the pulse oximeter 16. The cover 15, in this embodiment serves two purposes, to protect the light array 25 of the pulse oximeter 16 and also to house a photosensor 26 which measures light passing through a finger from the light array. As such, the photosensor 26 is facing toward a direction of light emitted from the light array 25. As noted above, in other embodiments, the photosensor may be on a same or adjacent surface as the light array, facing outward from the light array in a same direction as light emitted from the light array. The cover 15 may be spring loaded or otherwise have a flexible material to bias it in a closed position but which can be lifted open against the biasing force by the force of a finger. The cover 15 may also be removable, tethered, held in place by a magnet, and the like.

While the cover 15 is shown roughly the same width as the pulse oximeter 16 sensor area, it should be understood that in other embodiments, the cover 15 may be substantially wider, such as 50% wider, 100% wider, or may extend widthwise across the width of the case 10 rear wall 13. In a particular embodiment, the cover 15 may have a spring loaded accordion-like hinge which allows it to extend outwardly away from the case rear wall 13 when the pulse oximeter is in use, but be held closely to the cover 10 when not in use, thus minimizing the thickness of the cover 10. This minimizing of thickness may be important for convenience and to allow the case 10 to fit conveniently in, for example, a user's pocket, purse, and the like. Also on the rear wall is a display 14 which is in communication with a processor (not shown) and the display 14 is operable to show oxygen concentration (SpO2%) and pulse rate (PR bpm).

The cover may also be optimized for use under similar conditions that a smartphone is exposed to, including being dropped, extended time in a user's pocket or purse, having a hand over it during use, and the like. These acts all increase the expected loading and impact experienced by the cover 15, and also require the cover to be as thin and non-intrusive as possible. These problems are addressed in a number of ways. In one embodiment, the cover is made of a thin material so that it does not take up excessive space. The material may be selected to be sturdy and resilient, without being brittle, such as a strong plastic, metal reinforced plastic, metal and the like. In a particular embodiment, the hinge of the cover may be reinforced with thicker and more resilient material (not shown) than that found in a typical pulse oximeter due to the expected impact forces from a drop on the cover 15. The width thickness noted above may also improve durability and reduce thickness because a wider cover has a wider base to withstand twisting impacts and other lateral forces. In a further embodiment, the cover may have reinforcing arms which extend into the material of the rear wall to further reinforce the cover.

Similarly, the light array is also designed to withstand the above noted stresses expected of a smartphone case. The light array, in many embodiments, may have a resilient transparent or translucent cover such as a tempered glass or hard plastic to protect the light array components. This surface may also be easily cleanable to clean off dust, dirt and other materials which will cover the surface. Further still, the light array may be operable to be recalibrated using a programming of the processor in the event that extended use, an impact or other wear and tear can cause the components to become off alignment and/or uncalibrated.

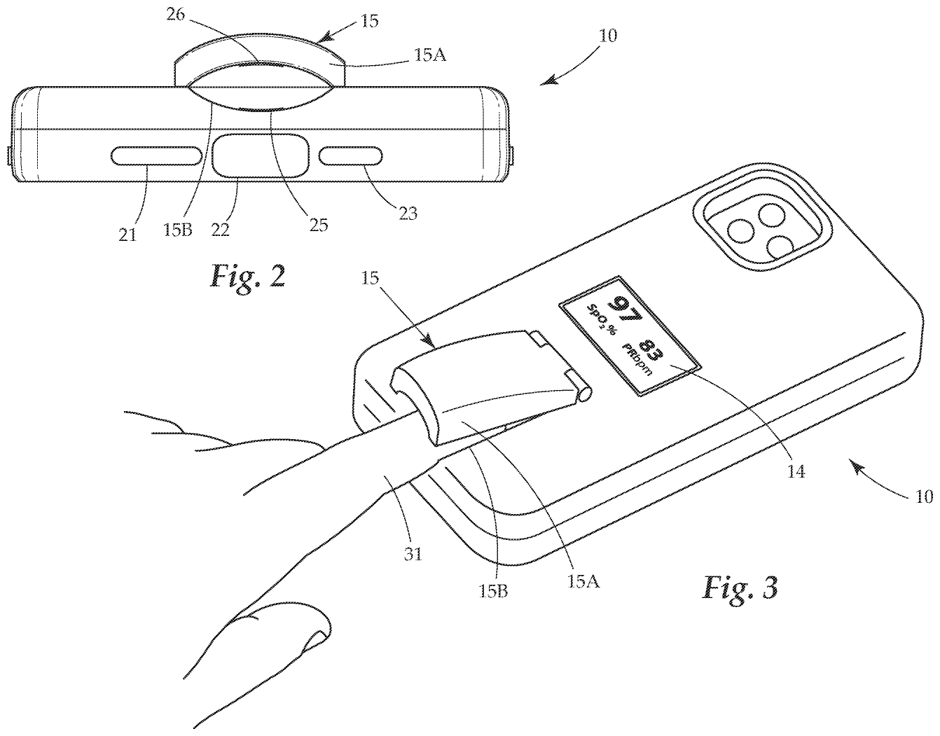

As can be seen in FIG. 2, the surface of the pulse oximeter 15 having the light array 25 has a concave shape 15B which corresponds to that of a human finger 31. The concave shape 15B allows for a flush and comfortable contact of the finger with the shaped surface 15B to closely contact the light array 25 leading to improved readings. Similarly, in this embodiment, the cover 15 has a concave shaped inner face 15A which also is adapted to receive a top of the human finger 31, again to achieve a good and non-interfered interface with the photosensor. Also visible in FIGS. 2 and 3 is the sidewall which extends from the rear wall to cover at least a part of an edge of the smartphone to removably engage the case with the smartphone. The sidewall at the bottom defines, in this embodiment, a speaker opening 23, a microphone opening 21, and a charge port opening 22.

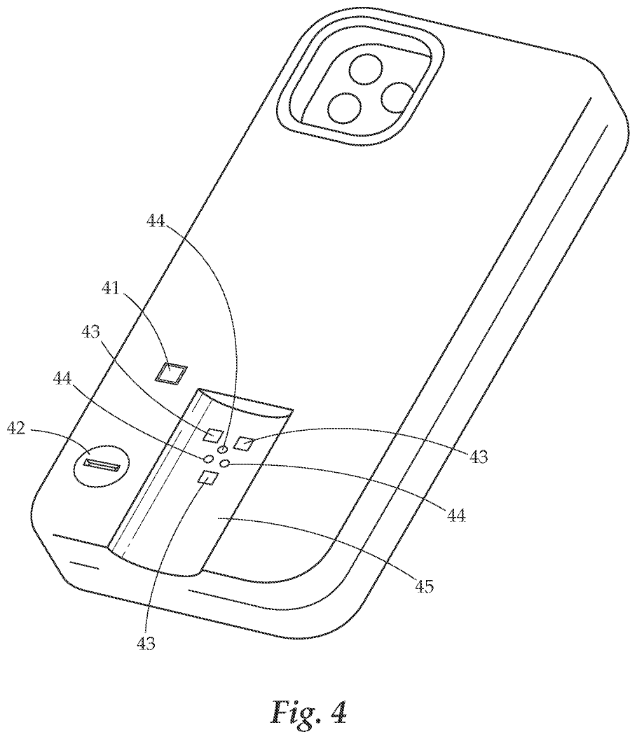

FIG. 4 provides a perspective view of an embodiment of the pulse oximeter which has a light array and photosensors on the same surface and facing outward or substantially outward (such that they may be angled relative to each other). The light array in this embodiment, utilizes three separate light sources 44, and has three photosensors 43. Of course, the number of light sources and photosensors may vary without straying from the scope of this invention. Also shown in this view are a processor 41 and battery 42 which are positioned within the case body. In one embodiment, the battery may be removable and replaceable, such as via an access cover on an inside or outside of the rear wall. In another embodiment, the battery may be rechargeable and permanently positioned on or in the case body.

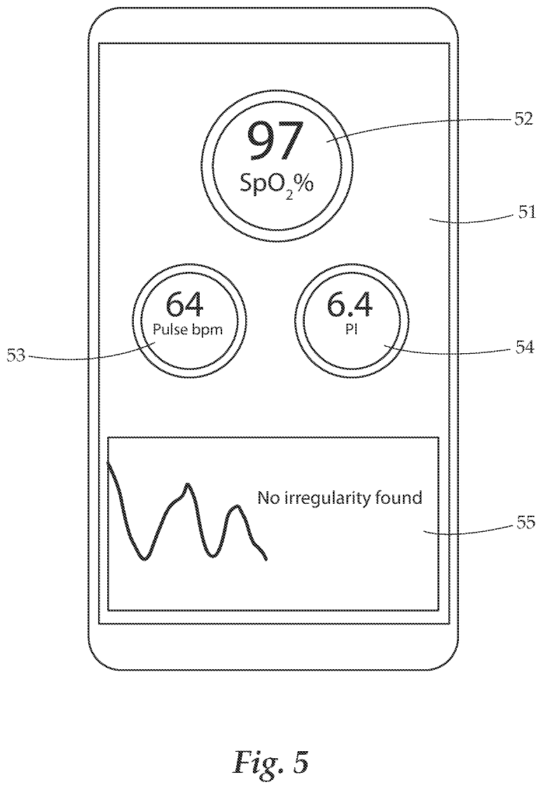

FIG. 5 shows a view of a user interface of an embodiment of a smartphone which is programmed with an application which works with the pulse oximeter of the case and its operational features. In a further embodiment, the smartphone and/or the pulse oximeter may be programmed to transfer data to a remote server and/or to a personal computer for downloading and/or uploading information. The pulse oximeter and its components may be in wired and/or wireless communication with the smartphone positioned in the case. In the embodiment shown, the smartphone and pulse oximeter are in wired or wireless communication such that a signal can be transmitted from the pulse oximeter and components to the smartphone. The smartphone is programmed, using the application, to display the signal as readouts of, in this embodiment, the display presents the oxygen concentration 52, pulse rate 53, and perfusion index 54. The user interface may also present an EKG readout 55 to measure heart function also using the pulse oximeter.

While several variations of the present disclosure have been illustrated by way of example in preferred or particular embodiments, it is apparent that further embodiments could be developed within the spirit and scope of the present disclosure, or the inventive concept thereof. However, it is to be expressly understood that such modifications and adaptations are within the spirit and scope of the present disclosure, and are inclusive, but not limited to the following appended claims as set forth.

What is claimed is:

1) A case for a smartphone comprising:
a rear wall and a sidewall portion extending from the rear wall to cover at least a part of an edge of the smartphone to removably engage the case with the smartphone; and
a pulse oximeter connected to the rear wall.

2) The case for a smartphone of claim 1 wherein the pulse oximeter further comprises:
a light array;
a sensor operable to measure light emitted from the light array;
a processor operable to receive an input from the sensor and calculate a blood oxygen saturation and pulse;
a battery in communication with the light array; and
a display in communication with the processor.

3) The case for a smartphone of claim 2 further comprising a smartphone positioned in the case such that the sidewalls cover at least the part of the edge of the smartphone, and wherein the display of the pulse oximeter is a display of the smartphone.

4) The case for a smartphone of claim 2 wherein the battery is positioned on or in the smartphone case.

5) The case for a smartphone of claim 2 wherein the processor is positioned on or in the smartphone case.

6) The case for a smartphone of claim 1 wherein the pulse oximeter further comprises a cover, the cover liftable away from a measurement surface of the pulse oximeter.

7) The case for a smartphone of claim 6 wherein the cover is biased by a spring or flexible material to a closed position, and liftable away from the measurement surface to an open position.

8) The case for a smartphone of claim 6 wherein the measurement surface comprises a light array.

9) The case for a smartphone of claim 8 wherein the cover comprises a sensor operable to measure light emitted from the light array.

10) The case for a smartphone of claim 1 wherein the pulse oximeter comprises a light array, and a sensor operable to measure light emitted from the light array.

11) The case for a smartphone of claim 10 wherein the sensor is on a cover opposite to the light array and facing toward a direction of light emitted from the light array.

12) The case for a smartphone of claim 10 wherein the sensor is on a same or adjacent surface as the light array and facing outward from the light array in a same direction as light emitted from the light array.

13) The case for a smartphone of claim 1 wherein the pulse oximeter has an inwardly curved portion to accommodate a curve of a human finger.

14) The case for a smartphone of claim 1 further comprising a display on an outer surface of the rear wall configured to display an oxygen saturation and pulse rate as measured from the pulse oximeter.

15) The case for a smartphone of claim 1 wherein the light array comprises at least a red emitting light and an infrared emitting light.

16) The case for a smartphone of claim 15 wherein the light array further comprises at least a green light.

17) The case for a smartphone of claim 1 wherein the pulse oximeter is in wireless communication with a smartphone positioned in the smartphone case.

18) The case for a smartphone of claim 1 wherein the sidewall defines a charge port opening, the charge port opening having a wiring body operable to direct a current flow to a battery of the pulse oximeter and to a battery of a smartphone when a smartphone is positioned in the case, the wiring body allowing a charging of both battery of the pulse oximeter and smartphone battery at the same time.

19) The case for a smartphone of claim 1 further comprising a wireless charging coil positioned in the rear wall, the wireless charging coil connected to a battery of the pulse oximeter and operable to charge the battery, wherein the wireless charging coil is smaller than a wireless charging coil of a smartphone intended to be placed in the case, so as to allow simultaneous wireless charging of a battery in the smartphone and the battery of the pulse oximeter.

20) A method for measuring a blood oxygen concentration comprising the steps of:
activating a pulse oximeter, the pulse oximeter stored on a smartphone case;
receiving a finger in or on the pulse oximeter;
calculating a blood oxygen concentration based on a reading from the pulse oximeter; and
presenting, on a display, information corresponding to a blood oxygen concentration recorded, the display being one of a display on the smartphone case or a display of a smartphone positioned in the smartphone case.
